(19) United States
(12) Patent Application Publication
(10) Pub. No.: US 2003/0082070 A1
(43) Pub. Date: May 1, 2003

(54) Sanitizing pouch and method of use therefor

(76) Inventors: Frank G. Liberto, San Antonio, TX (US); William F. Drees, Boerne, TX (US)

Correspondence Address: LAW OFFICES OF CHRISTOPHER L. MAKAY, 1634 Milam Building, 115 East Travis Street, San Antonio, TX, 78205, US

(21) Appl. No.: 10/280,620

(22) Filed: Oct. 25, 2002

Related U.S. Application Data
(60) Provisional application No. 60/339,411, filed on Oct. 26, 2001.

(51) Int. Cl. A61L 9/00; A62B 7/08; A61L 2/00

(52) U.S. Cl. 422/1; 422/28; 422/32; 422/120; 422/294; 422/300; 422/306

(57) ABSTRACT

A sanitizing pouch secures to a product package and includes a sanitizing agent disposed therein. A delivery device includes an inlet thereto and an outlet therefrom. A probe attaches to the inlet of the delivery device. The probe is adapted to puncture the sanitizing pouch and the product package, whereby the puncturing of the sanitizing pouch by the probe releases the sanitizing agent over the probe thereby sanitizing the probe prior to the puncturing of the product package by the probe. After flowing over the probe, the sanitizing agent flows into the delivery device thereby sanitizing the delivery device. The delivery device draws product from the product package for delivery via its outlet.

### BACKGROUND OF THE INVENTION

1. Field of the Invention

The present invention relates to food dispensing methods and apparatus, and more particularly, but not by way of limitation to a method and apparatus for dispensing packaged products with complete elimination or at least a minimum introduction of contamination to the product.

2. Description of the Related Art

In the fast paced work world, there is a great demand for quickly dispensed food and drink products. As this demand for speed and convenience increases, so does the need for sanitation systems that keep products preserved while waiting for distribution.

Modem methods of sterilization have greatly increased the amount of time a packaged product can remain in storage before expiring and becoming unusable. The long shelf life of modem packaged food products is the result of packaging methods used in production to sanitize both the product and the container.

One such method is aseptic packaging, where the package and the product are sterilized separately and then combined to form a completely sterile product environment. This environment, free of bacteria and mold, does not allow normal decomposition, or spoiling, of the product.

Packaging in a hot pack manner is another method. A product is heated to a particular temperature set by the FDA. This heating sterilizes the product by destroying any bacteria or molds present in the product. The product is then placed in a clean, but not necessarily sanitized packaged. Acidity levels in the product prevent growth of bacteria and molds within the packaging environment.

A third method, called retort, is typically used when canning products. Here a package is heated to an appropriate temperature or irradiated. A product is placed in the package and sealed therein. The product and packaging are then heated or irradiated to sanitize their interior environment.

All three of these methods have the advantage of keeping a product preserved and free from decomposition, or spoiling, for long periods. However, because of the cost of these methods, and especially the aseptic method, producing individual sized portions is not cost effective. Thus, in order for a sterilized product to be profitable for a distributor, it must be packaged in large containers and dispensed by retailers such as restaurants or mini-marts. This ultimately leads to a significant disadvantage. Once the product packaging is attached to a dispenser to allow dispensing of the product, contaminates can be reintroduced, significantly reducing the time the product is suitable for dispensing to consumers. Contaminates in this instance consist primarily of oxygen in the air reintroduced into to the product packaging, which restarts the natural process of decomposition or spoiling, or molds and bacteria in the air. Essentially, attaching a product package to the dispenser reintroduces contaminates.

Illustratively, a dispenser probe or valve used to break the seal of a product package and facilitate attachment of the package to a dispenser exposes the product in the product package to the air, thereby introducing contaminates into the package. Furthermore, the probe or valve is rarely washed and often comes in contact with dispenser users. Thus, every time the probe or valve is removed from one product package and coupled with another, it is exposed to airborne contaminates, which are then inserted with the probe or valve straight into the new sterilized package. This practice introduces increasing amounts of contaminates into the product, thereby significantly reducing the time the product remains dispensable to consumers. The current solution is simply to hope all product is dispensed before the requirement for disposal of unused product upon the expiration of a safe use time period, which is really no solution at all.

Accordingly, there is a long felt need for a method and apparatus that prevent the reintroduction of contaminates into a product when a product package is opened for dispensing.

### SUMMARY OF THE INVENTION

In accordance with the present invention, a sanitizing pouch secures to a product package and includes a sanitizing agent disposed therein. A delivery device includes an inlet thereto and an outlet therefrom. A probe attaches to the inlet of the delivery device. The probe is adapted to puncture the sanitizing pouch and the product package, whereby the puncturing of the sanitizing pouch by the probe releases the sanitizing agent over the probe thereby sanitizing the probe prior to the puncturing of the product package by the probe. After flowing over the probe, the sanitizing agent flows into the delivery device thereby sanitizing the delivery device. The delivery device draws product from the product package for delivery via its outlet. Alternatively, product may be displaced from the product package to the delivery device outlet.

The sanitizing pouch includes an outer container defining a pocket that contains the sanitizing agent therein. The outer container may include a first penetration point adapted for puncture by the probe. The outer container may further include a second penetration point positioned on the sanitizing pouch adjacent to the product package and adapted for puncture by the probe. The sanitizing pouch may include a wall that divides the outer container to separate a first chemical from a second chemical. Consequently, upon the puncturing of the wall by the probe, the first chemical combines with the second chemical to produce chemical heat that sanitizes the probe prior to the puncturing of the product package by the probe.

It is therefore an object of the present invention to provide a sanitizing pouch adapted to secure to a product package of a product delivery system.

It is another object of the present invention to provide a sanitizing pouch adapted to sanitize a probe of the product delivery system.

It is a further object of the present invention to provide a sanitizing pouch adapted to sanitize a delivery device of the product delivery system coupled to the probe.

Still other objects, features, and advantages of the present invention will become evident to those of ordinary skill in the art in light of the following.

### BRIEF DESCRIPTION OF THE DRAWINGS

FIG. 1 is a perspective view illustrating a sanitizing pouch according to a first embodiment.

FIG. 2 is a perspective view illustrating the sanitizing pouch according to the first embodiment.

FIG. 3 is a schematic diagram illustrating a product package with a sanitizing pouch attached thereto and a product delivery apparatus.

FIGS. 4a and 4b are schematic diagrams illustrating a product package with an alternative embodiment of a sanitizing pouch attached thereto.

FIG. 5 is a schematic diagram illustrating a product package with a sanitizing pouch attached thereto and example product delivery apparatus.

FIGS. 6-10 are schematic diagrams illustrating a product package with a sanitizing pouch attached thereto and the steps involved in coupling a product delivery apparatus to the product package.

FIG. 11 is a schematic diagram illustrating a product package with a sanitizing pouch attached thereto and a sterilized product delivery apparatus sealed in a sterile bag.

FIG. 12 is a schematic diagram illustrating a sterilized product delivery apparatus sealed in a sterile bag with a patch attached thereto and a product package.

### DETAILED DESCRIPTION OF THE PREFERRED EMBODIMENTS

The subject matter the applicant regards as the invention is particularly pointed out and distinctly claimed in the claims. The following descriptions are exemplary of preferred embodiments only, and are not intended to limit the scope of the invention in any way.

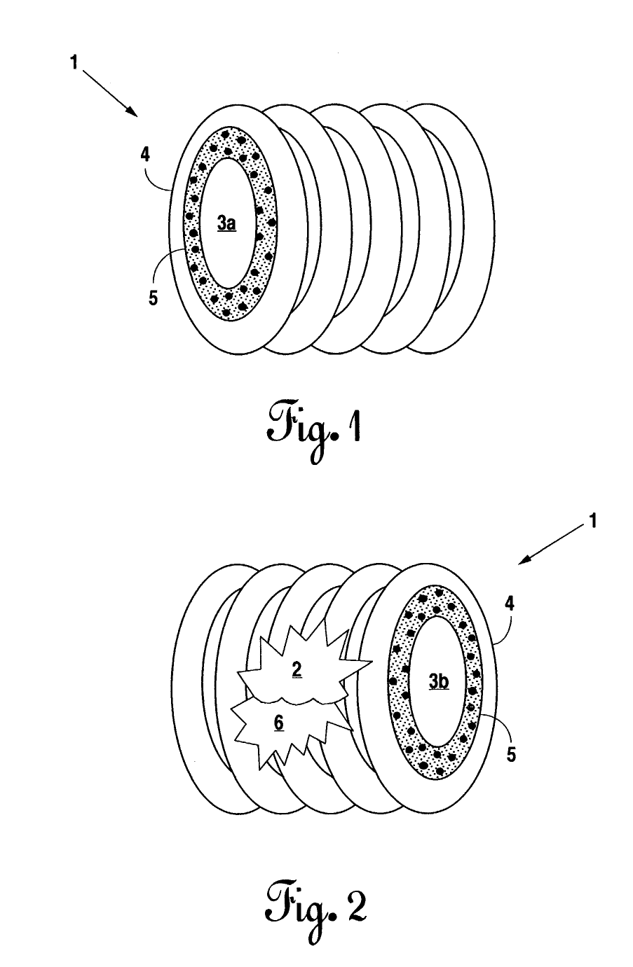

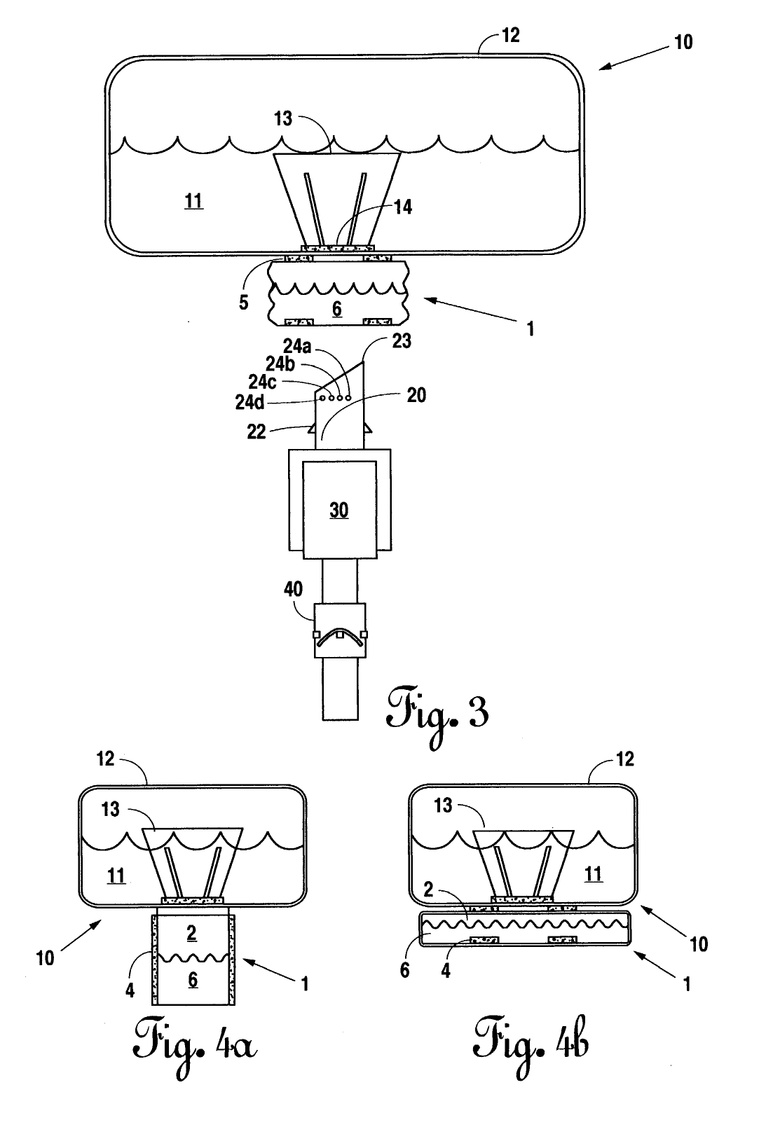

FIGS. 1 and 2 illustrate a sanitizing pouch 1, according to a first embodiment, shown in a collapsible, accordion design. The sanitizing pouch 1 includes an outer container 4 constructed of any suitable non-permeable material. Those of ordinary skill in the art will understand that the material composition of the outer container 4 depends upon the specific application for the sanitizing pouch 1 and includes such materials as plastic and the like. The outer container 4 defines a pocket 2 containing a sanitizing agent 6 therein. Those of ordinary skill in the art will recognize that the sanitizing agent 6 contained within pocket 2 depends upon the particular application for the sanitizing pouch 1 and includes such agents as nitrogen, hydrogen peroxide, chlorine solution, or alcohol.

Furthermore, in certain applications, chemically produced heat may perform the same function as a sanitizing agent 6. The outer container 4 would include a wall therein dividing the pocket 2 into two separate compartments. Each separate compartment would include a chemical or chemicals that react when combined to produce heat. Thus, upon the rupturing of the wall, the chemicals would combine in a reaction that produces heat sufficient for sanitization.

The sanitizing pouch 1 may include penetration points 3a and 3b on opposite ends that may comprise a slit or one or more perforated lines that allow for ease of penetration. An attaching means 5 may be located at one or both ends of the sanitizing pouch 1 to permit the securing of the sanitizing pouch 1 to a product package or to a dispensing device. Those of ordinary skill in the art will recognize many possible means for securing the sanitizing pouch 1 to a product package or dispensing device. Such means include: adherents or adhesives such as glue, sonic welding, threaded fittings, or an integral formation of the sanitizing pouch 1 with a product package.

FIGS. 4a and 4b illustrate certain alternative preferred embodiments of the sanitizing pouch 1, where like parts have been referenced with like numerals. Referring to FIG. 4a, an outer container 4 is a sleeve shaped to accommodate an elongated probe or punch. The sleeve is not necessarily collapsible. Further, the sleeve defines a pocket 2 having a sanitizing agent 6 therein. Referring to in FIG. 4b, an outer container 4 is a flexible bag suitable to accommodate a truncated probe or punch. The flexible bag defines a pocket 2 having a sanitizing agent 6 therein. The foregoing embodiments for the sanitizing pouch 1 merely represent examples of suitable shapes, sizes, designs, and the like. Accordingly, those of ordinary skill in the art will recognize many other embodiments having suitable shapes, sizes, designs, and the like, such as for example, a bellows design, balloon design, or a molded jelly sleeve design.

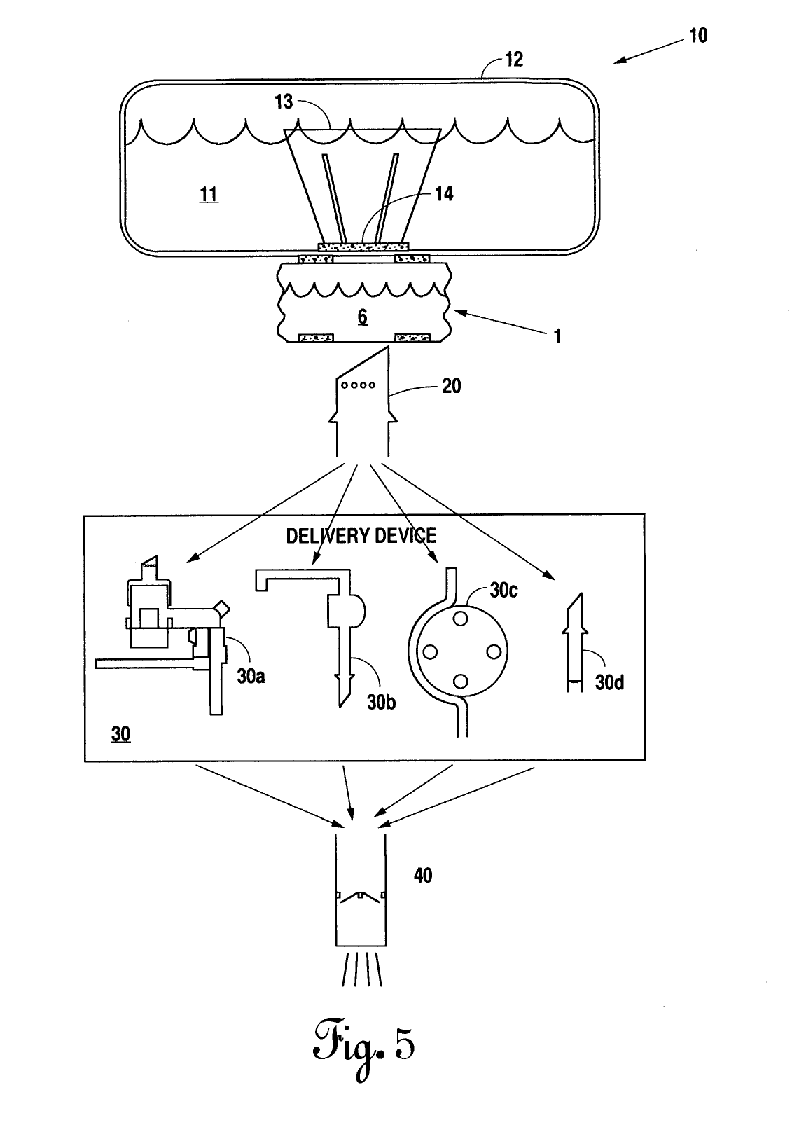

FIG. 3 illustrates the delivery of product from a product package 10. The product package 10 includes a container 12 with a product 11 disposed therein and a connector 13 suitable for coupling the product package 10 to a dispenser, delivery tube, and the like. The preferred connector 13 provides a snap fit with a dispenser, delivery tube, and the like; however, those of ordinary skill in the art will recognize many suitable alternative connectors, such as for example a threaded connector. The sanitizing pouch 1 attaches on the exterior to the product package 10 adjacent to the connector 13 utilizing an attaching means 5 as previously described, which, in the first preferred embodiment, is an adhesive that secures the sanitizing pouch 1 to the product package 10. Although the sanitizing pouch 1 may be secured to the product package 10 at any time, it is preferably attached thereto during the formation process for the product package 10.

The design of the product package 10 depends upon the desired application of the end user and may include many various shapes, sizes and materials, such as plastics, metals, composites, and the like. Flexible material designs for the product package 10 include a pouch, tote, bladder, or bag-in-box design. Rigid material designs for the product package 10 include a can, drum, tank, or barrel. Other designs include plastic devices such as jugs, bottles, or sealed cups. A laminate or a multi-layered synthetic material design is also feasible, once again, depending upon the desired application. FIG. 3 illustrates the product package 10 in a flexible bag configuration.

To facilitate the delivery of product from the product package 10, a probe 20 penetrates the sanitizing pouch 1 and the product package 10. The probe 20 includes a puncturing tip 23 that permits puncturing of the sanitizing pouch 1, preferably at penetration points 3a and 3b, as illustrated in FIGS. 1 and 2, and also the product package 10, preferably at a sealing lip 14 disposed on the connector 13. The sealing lip 14 is preferably constructed of a flexible, resilient material such that it conforms to the outer surface of the probe 20 when the probe 20 inserts into the connector 13. The sealing lip 14 forms a seal around the probe 20 and prevents product leakage or the introduction of contaminates from outside the product package 10. The probe 20 may include a securing surface 22, such as tines or detents, which engages the sealing lip 14 to the secure the probe 20 within the connector 13. After the insertion of the probe 20 into the product package 10, a suitable delivery device 30 draws product 11 from the product package 10 to a dispenser. A one-way valve 40 compartmentalizes the delivery device 30, the probe 20, and the product package 10 by preventing the backflow of the product 11. The one-way valve 40 therefore precludes the introduction of contaminates into the product package 10 through undesirable exposure of the product 11 to the outside environment. It should be understood by those of ordinary skill in the art that the location of the one-way valve 40 depends upon the desired configuration for compartmentalization and that multiple one-way valves may be included depending upon the degree of compartmentalization required. Illustratively, the one-way valve 40 may be placed prior to the delivery device 30 or a second one-way valve may be placed prior to the delivery device 30.

During the insertion of the probe 20, the sanitizing agent 6 washes over the probe 20 as the probe 20 passes through the sanitizing pouch 1 prior to the probe 20 penetrating the product package 10, thereby sanitizing the probe 20 to prohibit the probe 20 from introducing contaminates into the product package 10. The probe 20 may include apertures 24a-d that serve to release air captured in the probe 20 as it enters the sanitizing pouch 1. The apertures 24a-d further aid in dispersing the sanitizing agent 6 onto the exterior of the probe 20, thereby sanitizing the exterior of the probe 20 as well. After the probe 20 passes through the sanitizing pouch 1, the puncturing tip 23 contacts the product package 10 to permit the penetrating of the product package 10 by the probe 20. The probe 20 passes through the exterior of the product package 10 and engages the connector 13 to couple the probe 20 with the product package 10. The sanitizing agent 6 in washing over the probe 20 while it punctures the product package 10 encapsulates the probe 20 to prevent the exposure of the product 11 to the surrounding air as the probe 20 enters and couples with the product package 10.

With the probe 20 coupled to the product package 10, the sanitizing agent 6 travels into the delivery device 30. Thus, upon the activation of the delivery device 30, the sanitizing agent 6 passes through the delivery device 30 and the one-way valve 40, thereby sanitizing the delivery device 30 and the one-way valve 40 to prevent the delivery device 30 and the one-way valve 40 from introducing contaminates into the product package 10. After the sanitizing of the delivery device 30 and the one-way valve 40, the one-way valve 40 precludes the introduction of contaminates through backflow prevention as previously described. In employing the sanitizing pouch 1, it should be understood that a certain amount of initial product 11 is used to flush the sanitizing agent 6 through the delivery device 30 and the one-way valve 40, which requires the disposal of that initial amount of product 11. Once the probe 20 has been inserted into product package 10 and the delivery device 30 and the one-way valve 40 flushed, the delivery device 30 when activated draws the product 11 from the product package 10 and delivers the product 111 to a dispenser, delivery tube, and the like for dispensing into a suitable container.

The inclusion of a sanitizing pouch 1 on a product package 10 solves the problem associated with the use of sterile packaging, which permits the product package 10 to be large in size. Essentially, the buffering provided to a product 11 by a sanitizing agent 6 as well as the sanitizing of a probe 20, a delivery device 30, and a one-way valve 40 prevents the introduction of contaminates into a product package 10. Further, a one-way valve 40 prevents product 111 within a probe 20 and a delivery device 30 from being exposed to the outside environment, which again prevents the introduction of contaminates into a product package 10. Accordingly, a sanitizing pouch 1 provides an opened product package 10 with substantially the same shelf life as an unopened product package 10.

FIG. 5 illustrates example embodiments for the delivery device 30. In a first embodiment, the delivery device 30 may be a reciprocating piston pump 30a. In a second embodiment, the delivery device 30 may be a manual suction pump 30b, such as that disclosed in U.S. Pat. No. 5,452,826. In a third embodiment, the delivery device 30 may be a peristaltic pump. Although the present invention has been disclosed as including a delivery device 30 between the product package 10 and the one-way valve 40, those of ordinary skill in the art will recognize that a delivery device may be included at any point in a dispenser system sufficient to facilitate delivery of product from the product package 10. Illustratively, the product package 10 could be pressurized to facilitate product delivery or could include a piston to force product from the product package 10. Accordingly, a probe and one-way valve would be included separate from the delivery device as illustrated in FIG. 5 and referenced with numeral 30d.

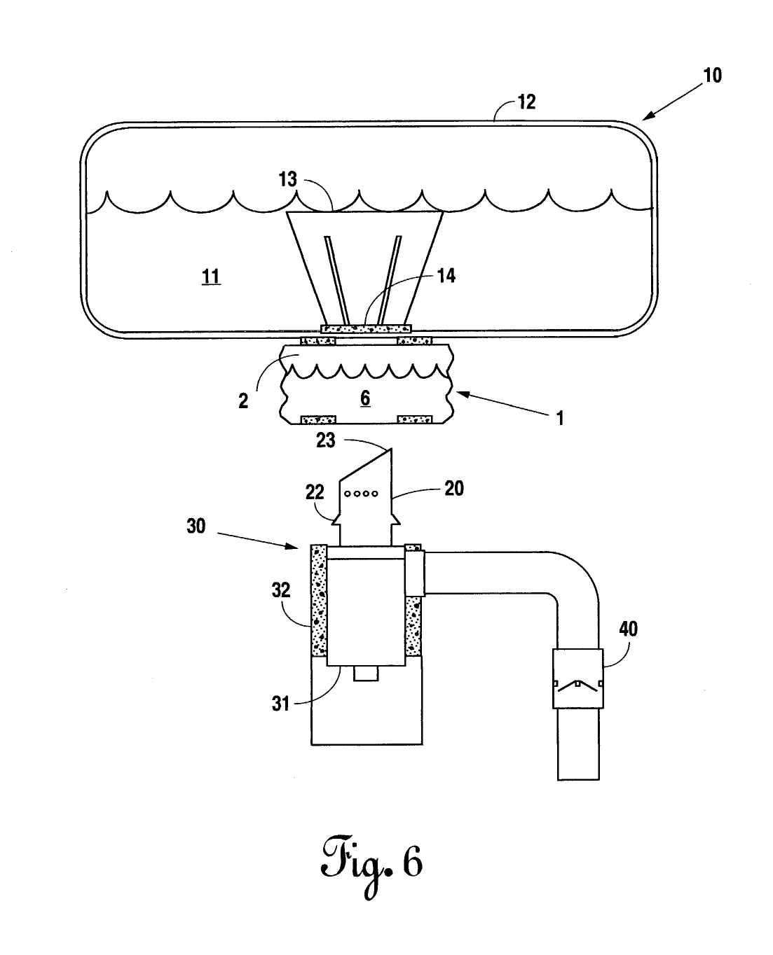

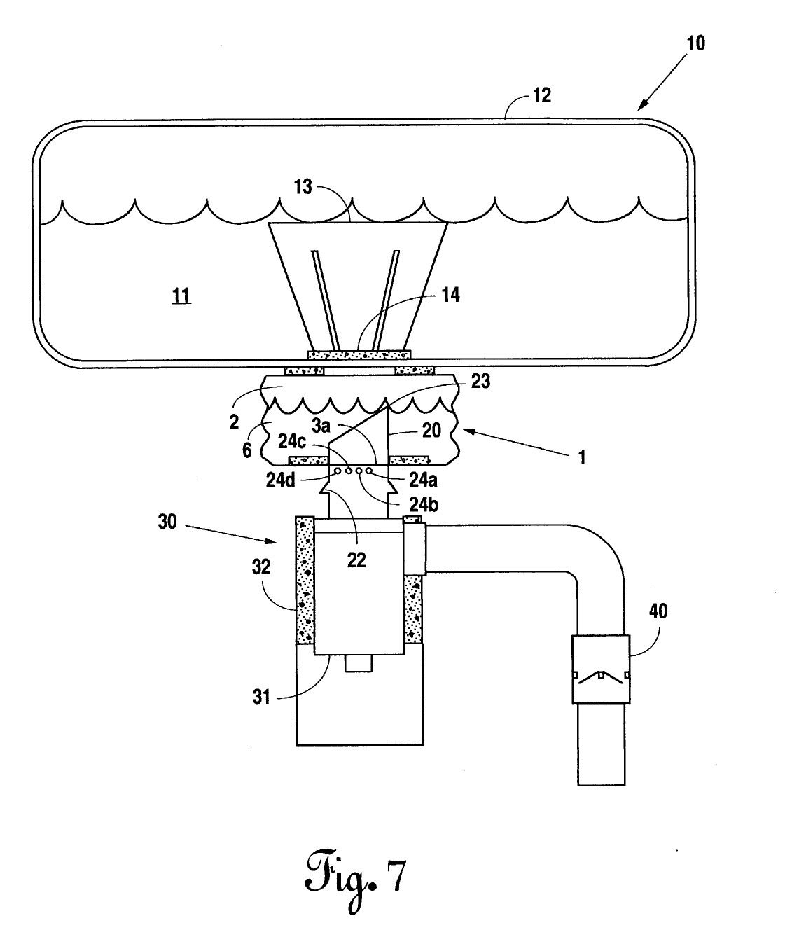

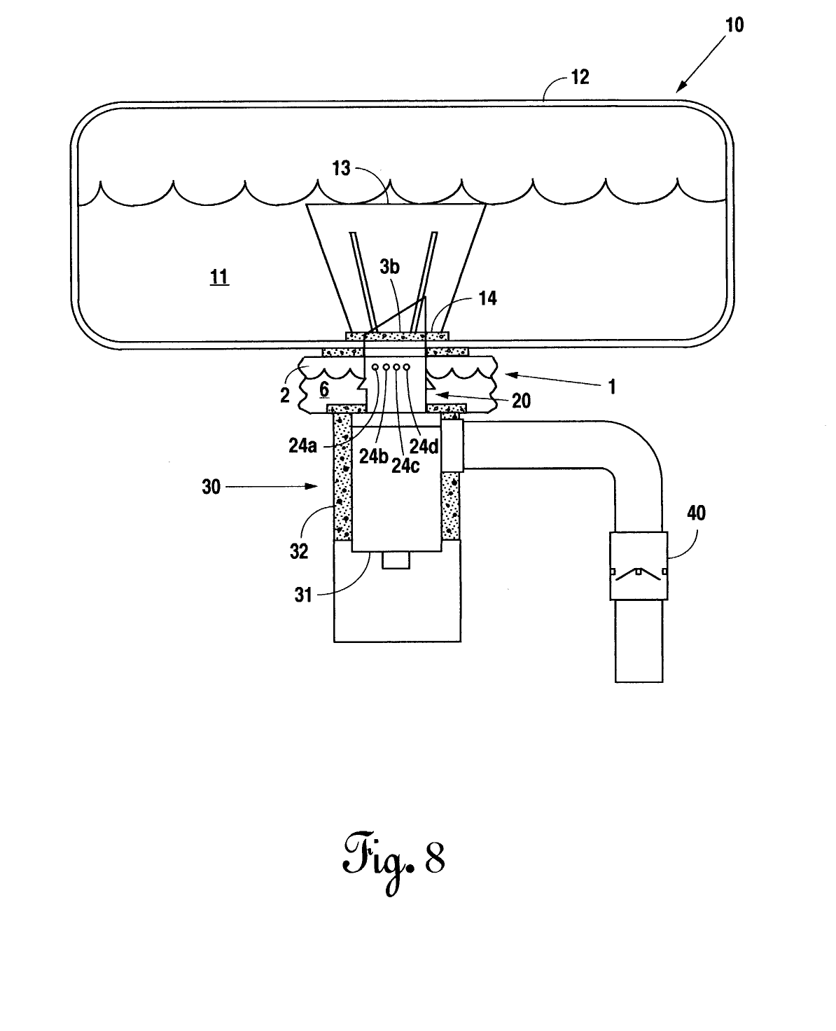

In order to aid in the understanding of the present invention, the operation of a reciprocating piston pump 31 as the delivery device 30 is disclosed herein with reference to FIGS. 6-10. The reciprocating piston pump 31 is suitable to pump either pre-mixed or stand alone products or post-mix products formed from concentrates. For post-mix products formed from concentrates, the reciprocating piston pump 31 would attach to a mixing chamber including a port that connects to a diluent source (e.g., water). The reciprocating piston pump 31 would thus pump product concentrate into the mixing chamber for combination with the diluent to form a final product.

FIG. 6 illustrates a starting position, or a first step, of a sanitizing process for a probe 20; a delivery device 30, which is a reciprocating piston pump 31 in this particular application; one-way valve 40; and associated lines. A product package 10 contains a container 12, a product 11, and a connector 13, which is a receptor designed to receive, secure and stabilize the probe 20 as previously described with reference to FIG. 3. The connector 13 further prevents the container 12 from collapsing around the probe 20 when the reciprocating piston pump 31 creates a vacuum in the container 12, which could result in the restriction of product flow from the product package 10. A sanitizing pouch 1 containing a sanitizing agent 6 within a pocket 2 is secured to the product package 10 as previously described with reference to FIGS. 1-3.

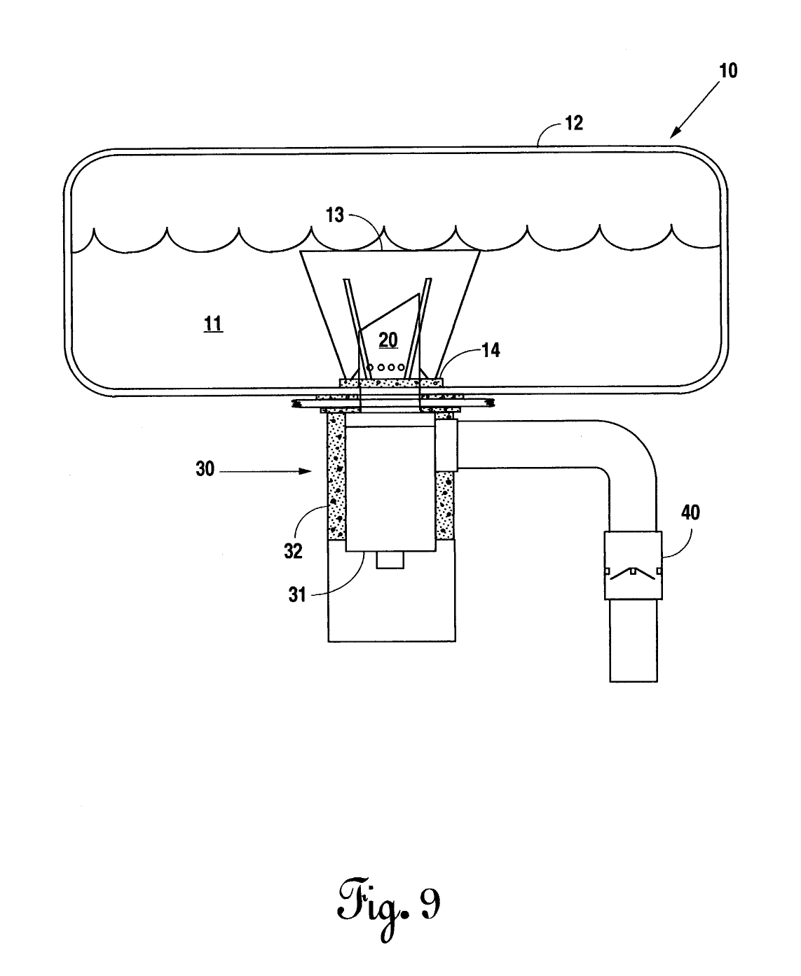

FIG. 7 illustrates a second step of the sanitizing process, whereby a puncturing tip 23 of the probe 20 punctures through one penetration point 3a of the sanitizing pouch 1 to allow the probe 20 to enter the pocket 2 and contact the sanitizing agent 6. The probe 20 may include apertures 24a-d that serve to release air captured in the probe 20 as it enters the sanitizing pouch 1. The apertures 24a-d further aid in dispersing the sanitizing agent 6 onto the exterior of the probe 20, thereby sanitizing the exterior of the probe 20 as well. The sanitizing agent 6 sanitizes the interior and exterior of the probe 20 and flows into the reciprocating piston pump 31 via the probe 20.

FIG. 8 illustrates a third step of the sanitizing process, whereby, when the probe 20 reaches the opposite end of the sanitizing pouch 1, the puncturing tip 23 punctures the second penetration point 3b and also penetrates the product package 10. The probe 20 passes through a sealing lip 14 on the connector 13 and couples with the connector 13 as previously described with reference to FIG. 3. The sanitizing agent 6 in washing over the probe 20 while it punctures the product package 10 encapsulates the probe 20 to prevent the exposure of the product 11 to the surrounding air as the probe 20 enters and couples with the product package 10. The diameters of the penetration points 3a and 3b are such that when the probe 20 enters the sanitizing pouch 1 there is no exterior leakage through the breech. Further, the sanitizing pouch 1, which is the first embodiment of a collapsible accordion design, collapses as a pump casing 32 of the reciprocating pump 31 applies pressure to the end opposite from the product package 10.

FIG. 9 illustrates a fourth step of the sanitizing process, whereby a securing surface 22, such as one or more tines or detents, displaces resilient material of the sealing lip 14 as the probe 20 enters product the product package 10. When the resilient material of the sealing lip 14 reshapes to form an airtight seal around the outer surface of the probe 20, the securing surface 22 seats on the interior surface of the sealing lip 14, thereby preventing the removal of the probe 20. With the probe coupled with the connector 13, product 11 can begin to flow through the probe 20 and into the reciprocating piston pump 31 for dispensing into a suitable container.

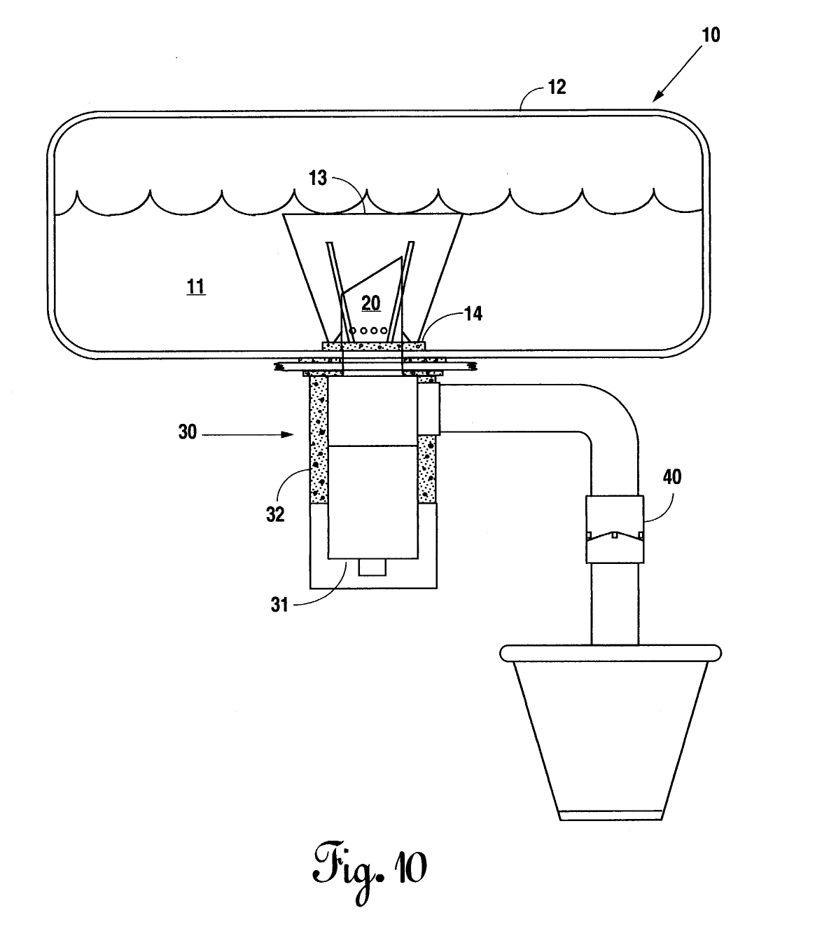

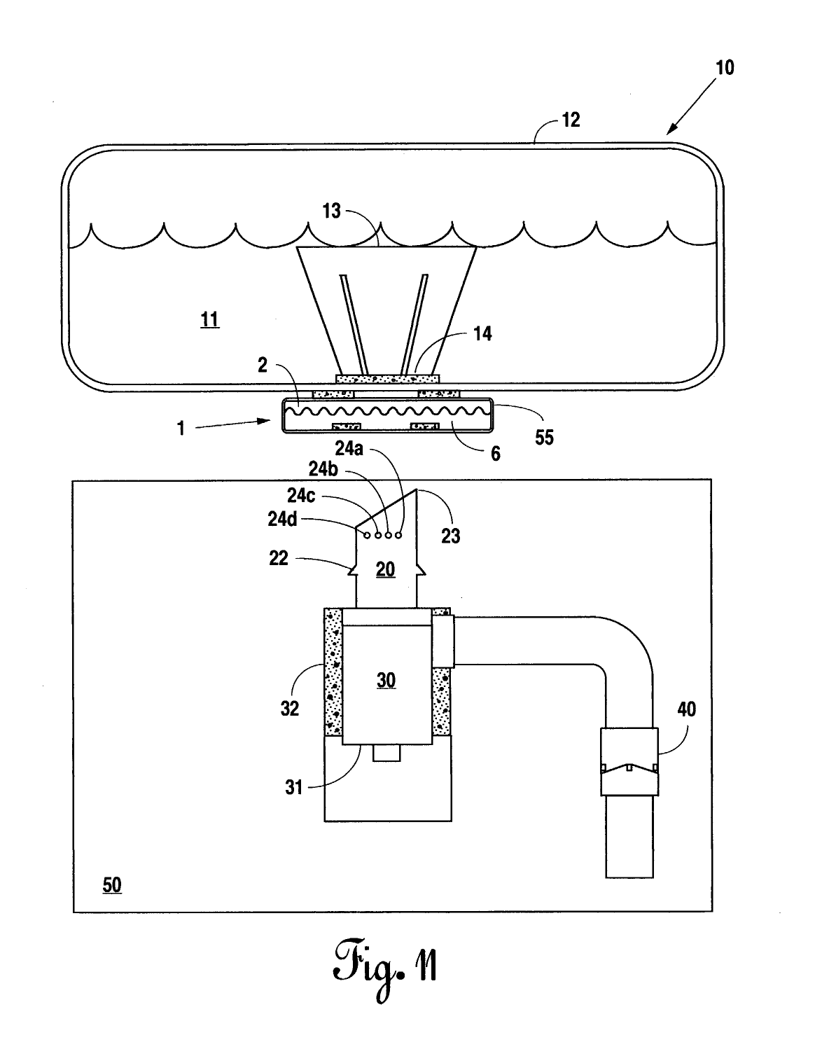

FIG. 10 illustrates a final step of the sanitizing process, whereby, upon the activation of the reciprocating piston pump 31, the sanitizing agent 6 passes through the reciprocating piston pump 31 and the one-way valve 40, thereby sanitizing the reciprocating piston pump 31 and the one-way valve 40 to prevent the reciprocating piston pump 31 and the one-way valve 40 from introducing contaminates into the product package 10. After the sanitizing of the reciprocating piston pump 31 and the one-way valve 40, the one-way valve 40 precludes the introduction of contaminates through backflow prevention as previously described with reference to FIG. 3. It should be understood by those of ordinary skill in the art that the location of the one-way valve 40 depends upon the desired configuration for compartmentalization and that multiple one-way valves may be included depending upon the degree of compartmentalization required. Illustratively, the one-way valve 40 may be placed prior to the reciprocating piston pump 31 or a second one-way valve may be placed prior to the reciprocating piston pump 31.

In employing the sanitizing pouch 1, it should be understood that a certain amount of initial product 11 is used to flush the sanitizing agent 6 through the reciprocating piston pump 31 and the one-way valve 40, which requires the disposal of that initial amount of product 11. After the flushing of the reciprocating piston pump 31 and the one-way valve 40, the reciprocating piston pump 31 when activated draws the product 11 from the product package 10 and delivers the product 11 to a dispenser for dispensing into a suitable container.

FIG. 11 is a schematic diagram illustrating a product package 10 with a sanitizing pouch 1 attached thereto and a sterilized a probe 20, a delivery device 30, one-way valves 40, and associated lines sealed in a bag 50. The probe 20, the delivery device 30, the one-way valve 40, and the associated lines are sealed in the bag 50 and sterilized using any of several well-known sterilization techniques, such as irradiation. This ensures the probe 20, the delivery device 30, the one-way valve 40, and the associated lines do not introduce contaminates into the product package 10 upon coupling thereto. Nevertheless, although the probe 20, the delivery device 30, the one-way valve 40, the associated lines, and the interior of the bag 50 are sterile, the exterior of the bag 50 is not and could introduce contaminates onto the probe 20 as the probe 20 inserts into the product package 10. Accordingly, the product package 10 includes a sanitizing pouch 1 attached thereto, which in this instance is a flexible bag 55 containing a sanitizing agent 6 within a pocket 2 as previously shown and described with reference to FIG. 4b.

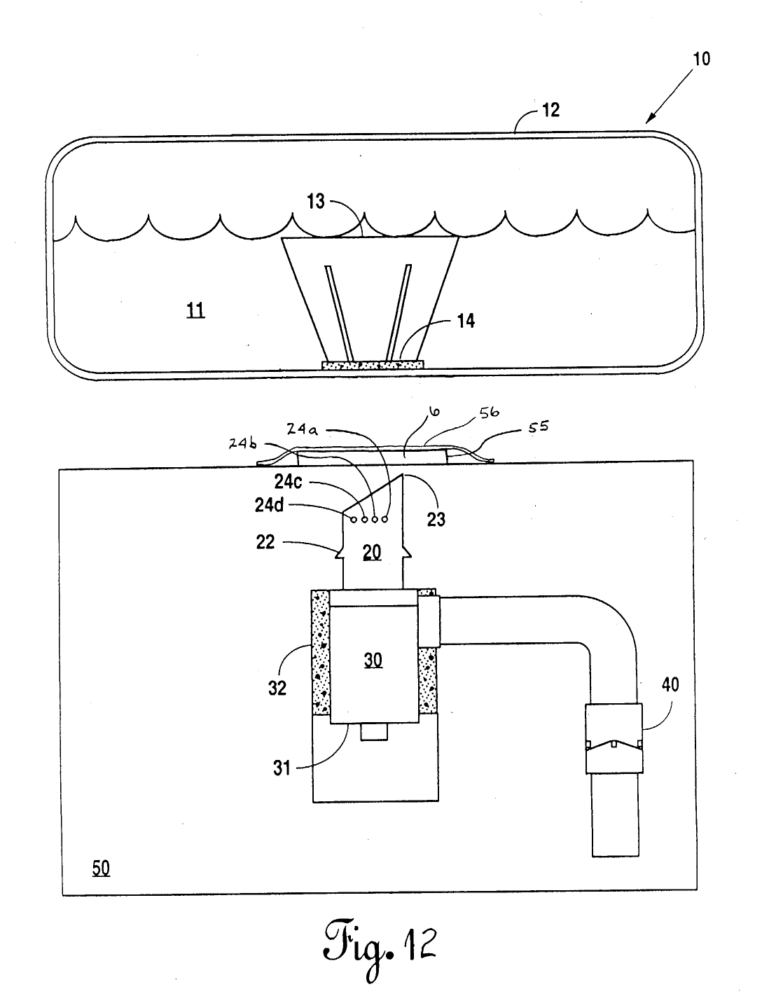

Alternatively, as illustrated in FIG. 12, the bag 50 may include a patch 55 or patches adhesively secured thereto. The patch 55 is impregnated with the sanitizing agent 6 and the adhesive securing the patch 55 to the bag 50 is also formulated from or includes the sanitizing agent 6. Furthermore, the patch 55 includes an adhesive on the side opposite the bag 50 that is also formulated from or includes the sanitizing agent 6. The patch 55 accordingly includes a cover 56 that protects the patch 55 and the adhesive from contamination. Although the patch 55 is disclosed as secured to the bag 50, those of ordinary skill in the art will recognize that the patch 55 may be secured to the product package 10.

The bag 50 is placed adjacent the sanitizing pouch 1, and a puncturing tip 23 of the probe 20 punctures through the bag 50 and the sanitizing pouch 1 to allow the probe 20 to enter the pocket 2 and contact the sanitizing agent 6. Alternatively, the cover 56 is removed from the patch 55, and the bag 50 is secured adjacent the container 12 so that a puncturing tip 23 of the probe 20 punctures through the bag 50 and the patch 55 to contact the sanitizing agent 6 of the patch 55. The sanitizing agent 6 flows into the probe 20 and also along the exterior of the bag 50 to sanitize both the probe 20 and the exterior of the bag 50. The probe 20 may include apertures 24a-d that serve to release air captured in the probe 20 as it enters the sanitizing pouch 1. The apertures 24a-d further aid in dispersing the sanitizing agent 6 onto the exterior of the probe 20, thereby sanitizing the exterior of the probe 20 as well. The sanitizing agent 6 further flows into the delivery device 30 via the probe 20.

When the probe 20 reaches the opposite end of the sanitizing pouch 1, the puncturing tip 23 exits the sanitizing pouch 1 and punctures the product package 10. The probe 20 passes through a sealing lip 14 on a connector 13 and couples with the connector 13 as previously described with reference to FIG. 3. With the probe coupled with the connector 13, product 11 can begin to flow through the probe 20 and into the delivery device 30 for dispensing into a suitable container.

Upon the activation of the delivery device 30, the sanitizing agent 6 passes through the delivery device 30 and the one-way valve 40, thereby sanitizing the delivery device 30 and the one-way valve 40 to prevent the reciprocating piston pump 31 and the one-way valve 40 from introducing contaminates into the product package 10. After the sanitizing of the delivery device 30 and the one-way valve 40, the one-way valve 40 precludes the introduction of contaminates through backflow prevention as previously described with reference to FIG. 3. In employing the sanitizing pouch 1, it should be understood that a certain amount of initial product 11 is used to flush the sanitizing agent 6 through the delivery device 30 and the one-way valve 40, which requires the disposal of that initial amount of product 11. After the flushing of the delivery device 30 and the one-way valve 40, the delivery device 30 when activated draws the product 11 from the product package 10 and delivers the product 11 to a dispenser for dispensing into a suitable container.

Although the present invention has been described in terms of the foregoing embodiments, such description has been for exemplary purposes only and, as will be apparent to those of ordinary skill in the art, many alternatives, equivalents, and variations of varying degrees will fall within the scope of the present invention. That scope, accordingly, is not to be limited in any respect by the foregoing description; rather, it is defined only by the claims that follow.

What is claimed is:

1. An apparatus for sanitizing a product delivery system, comprising:
a sanitizing pouch securable to a product package; and
a sanitizing agent disposed within the sanitizing pouch, whereby, upon the puncturing of the sanitizing pouch by the probe, the sanitizing agent contacts the probe thereby sanitizing the probe prior to the entry of the probe into the product package.

2. The apparatus for sanitizing a product delivery system according to claim 1, wherein, after contacting the probe, the sanitizing agent flows into a delivery device coupled with the probe thereby sanitizing the delivery device.

3. The apparatus for sanitizing a product delivery system according to claim 2, wherein the sanitizing agent flows into a one-way valve of the delivery device thereby sanitizing the one-way valve.

4. The apparatus for sanitizing a product delivery system according to claim 1, wherein the sanitizing pouch comprises an outer container adapted to hold the sanitizing agent therein.

5. The apparatus for sanitizing a product delivery system according to claim 1, wherein the sanitizing pouch defines a pocket that contains the sanitizing agent therein.

6. The apparatus for sanitizing a product delivery system according to claim 1, wherein the sanitizing pouch includes a first penetration point adapted for puncture by the probe.

7. The apparatus for sanitizing a product delivery system according to claim 6, wherein the sanitizing pouch includes a second penetration point positioned on the sanitizing pouch adjacent to the product package and adapted for puncture by the probe.

8. The apparatus for sanitizing a product delivery system according to claim 4, wherein the sanitizing pouch includes a wall that divides the outer container to separate a first chemical from a second chemical, whereby the puncturing of the wall by the probe combines the first chemical with the second chemical thereby producing a chemical heat that sanitizes the probe prior to the puncturing of the product package by the probe.

9. The apparatus for sanitizing a product delivery system according to claim 1, wherein the sanitizing pouch comprises a collapsible accordion configuration.

10. The apparatus for sanitizing a product delivery system according to claim 1, wherein the sanitizing pouch comprises a sleeve.

11. The apparatus for sanitizing a product delivery system according to claim 1, wherein the sanitizing pouch comprises a flexible bag.

12. The apparatus for sanitizing a product delivery system according to claim 1, wherein the probe includes an aperture that releases air captured in the probe as the probe punctures the sanitizing pouch.

13. The apparatus for sanitizing a product delivery system according to claim 1, wherein the probe includes an aperture that disperses sanitizing agent to an exterior surface of the probe during the puncturing of the sanitizing pouch by the probe thereby sanitizing the exterior surface of the probe prior to the puncturing of the product package by the probe.

14. An apparatus for delivering a product, comprising:
a product package;
a sanitizing pouch securable to the product package, the sanitizing pouch including a sanitizing agent disposed therein;
a delivery device including an inlet thereto and an outlet therefrom;
a probe coupled with the inlet of the delivery device, the probe adapted to puncture the sanitizing pouch and the product package, whereby the puncturing of the sanitizing pouch by the probe releases the sanitizing agent over the probe thereby sanitizing the probe prior to the puncturing of the product package by the probe.

15. The apparatus for delivering a product according to claim 14, wherein the product package, comprises:
a container adapted to house a product; and
a connector mounted with the container and adapted to engage the probe to couple the probe with the container.

16. The apparatus for delivering a product according to claim 15, wherein the sanitizing pouch secures to the container in a position adjacent to the connector.

17. The apparatus for delivering a product according to claim 14, wherein the sanitizing pouch comprises an outer container adapted to hold the sanitizing agent therein.

18. The apparatus for delivering a product according to claim 14, wherein the sanitizing pouch defines a pocket that contains the sanitizing agent therein.

19. The apparatus for delivering a product according to claim 14, wherein the sanitizing pouch includes a first penetration point adapted for puncture by the probe.

20. The apparatus for delivering a product according to claim 19, wherein the sanitizing pouch includes a second penetration point positioned on the sanitizing pouch adjacent to the product package and adapted for puncture by the probe.

21. The apparatus for delivering a product according to claim 17, wherein the sanitizing pouch includes a wall that divides the outer container to separate a first chemical from a second chemical, whereby the puncturing of the wall by the probe combines the first chemical with the second chemical thereby producing a chemical heat that sanitizes the probe prior to the puncturing of the product package by the probe.

22. The apparatus for delivering a product according to claim 14, wherein the sanitizing pouch comprises a collapsible accordion configuration.

23. The apparatus for delivering a product according to claim 14, wherein the sanitizing pouch comprises a sleeve.

24. The apparatus for delivering a product according to claim 14, wherein the sanitizing pouch comprises a flexible bag.

25. The apparatus for delivering a product according to claim 14, wherein the probe includes an aperture that releases air captured in the probe as the probe punctures the sanitizing pouch.

26. The apparatus for delivering a product according to claim 14, wherein the probe includes an aperture that disperses sanitizing agent to an exterior surface of the probe during the puncturing of the sanitizing pouch by the probe thereby sanitizing the exterior surface of the probe prior to the puncturing of the product package by the probe.

27. The apparatus for delivering a product according to claim 14, wherein, after flowing over the probe, the sanitizing agent flows into the delivery device thereby sanitizing the delivery device.

28. The apparatus for delivering a product according to claim 14, wherein the delivery device includes a one-way valve disposed in the outlet therefrom.

29. The apparatus for delivering a product according to claim 28, wherein, upon initial activation of the delivery device, the sanitizing agent flows through the delivery device and the one-way valve thereby sanitizing the delivery device and the one-way valve.

30. The apparatus for delivering a product according to claim 14, wherein the delivery device includes a one-way valve disposed in the inlet thereto.

31. The apparatus for delivering a product according to claim 30, wherein, upon initial activation of the delivery device, the sanitizing agent flows through the one-way valve and the delivery device thereby sanitizing the one-way valve and the delivery device.

32. The apparatus for delivering a product according to claim 14, wherein the delivery device includes a first one-way valve disposed in the inlet thereto and a second one-way valve disposed in the outlet therefrom.

33. The apparatus for delivering a product according to claim 32, wherein, upon initial activation of the delivery device, the sanitizing agent flows through the first one-way valve, the delivery device, and the second one-way valve thereby sanitizing the first one-way valve, the delivery device, and the second one-way valve.

34. The apparatus for delivering a product according to claim 14, wherein the delivery device comprises reciprocating piston pump.

35. The apparatus for delivering a product according to claim 14, wherein the delivery device comprises a suction pump.

36. The apparatus for delivering a product according to claim 14, wherein the delivery device comprises a manual suction pump.

37. The apparatus for delivering a product according to claim 14, wherein the delivery device comprises a peristaltic pump.

38. The apparatus for delivering a product according to claim 14, wherein the product package includes a piston adapted to force product from the product package.

39. The apparatus for delivering a product according to claim 38, wherein the delivery device comprises an elongated probe including a one-way valve disposed in an outlet therefrom.

40. The apparatus for delivering a product according to claim 14, wherein the probe and the delivery device are sterilized and sealed in a sterilized bag.

41. The apparatus for delivering a product according to claim 40, wherein the probe is adapted to puncture the bag, the sanitizing pouch, and the product package, whereby the puncturing of the bag and the sanitizing pouch by the probe releases the sanitizing agent over the probe and the bag thereby sanitizing the bag and the probe prior to the puncturing of the product package by the probe.

42. A method for sanitizing a product delivery system, comprising:
securing a sanitizing pouch to a product package, the sanitizing pouch including a sanitizing agent disposed therein; and
puncturing the sanitizing pouch with the probe to release the sanitizing agent over the probe thereby sanitizing the probe prior to the entry of the probe into the product package.

43. The method for sanitizing a product delivery system according to claim 42, further comprising flowing the sanitizing agent into a delivery device coupled with the probe thereby sanitizing the delivery device.

44. The method for sanitizing a product delivery system according to claim 43, further comprising flowing the sanitizing agent into a one-way valve of the delivery device thereby sanitizing the one-way valve.

45. The method for sanitizing a product delivery system according to claim 42, further comprising:
dividing the sanitizing pouch with a wall to separate a first chemical from a second chemical;
puncturing the wall with the probe to combine the first chemical with the second chemical; and
producing a chemical heat that sanitizes the probe prior to the puncturing of the product package by the probe.

46. The method for sanitizing a product delivery system according to claim 42, further comprising releasing air captured in the probe during the puncturing of the sanitizing pouch by the probe.

47. The method for sanitizing a product delivery system according to claim 42, further comprising dispersing the sanitizing agent to an exterior surface of the probe during the puncturing of the sanitizing pouch by the probe thereby sanitizing the exterior surface of the probe prior to the puncturing of the product package by the probe.

48. A method for delivering a product, comprising:
securing a sanitizing pouch to a product package, the sanitizing pouch including a sanitizing agent disposed therein;
providing a delivery device with a probe attached thereto; and
puncturing the sanitizing pouch with the probe to release the sanitizing agent over the probe thereby sanitizing the probe prior to the puncturing of the product package by the probe.

49. The method for delivering a product according to claim 48, further comprising puncturing the product package with the probe.

50. The method for sanitizing a product delivery system according to claim 49, further comprising flowing the sanitizing agent into the delivery device thereby sanitizing the delivery device.

51. The method for delivering a product according to claim 50, further comprising drawing product from the product package utilizing the delivery device.

52. The method for delivering a product according to claim 50, further comprising displacing product from the product package to the delivery device.

53. The method for sanitizing a product delivery system according to claim 50, further comprising flowing the sanitizing agent into a one-way valve disposed in an outlet of the delivery device thereby sanitizing the one-way valve.

54. The method for delivering a product according to claim 53, further comprising delivering the product from the delivery device via the one-way valve disposed in the outlet of the delivery device.

55. The method for sanitizing a product delivery system according to claim 49, further comprising flowing the sanitizing agent into a one-way valve disposed in an inlet of the delivery device thereby sanitizing the one-way valve.

56. The method for sanitizing a product delivery system according to claim 55, further comprising flowing the sanitizing agent into the delivery device thereby sanitizing the delivery device.

57. The method for delivering a product according to claim 56, further comprising drawing product from the product package utilizing the delivery device.

58. The method for delivering a product according to claim 56, further comprising displacing product from the product package to the delivery device.

59. The method for sanitizing a product delivery system according to claim 48, further comprising releasing air captured in the probe during the puncturing of the sanitizing pouch by the probe.

60. The method for delivering a product according to claim 48, further comprising dispersing the sanitizing agent to an exterior surface of the probe during the puncturing of the sanitizing pouch by the probe thereby sanitizing the exterior surface of the probe prior to the puncturing of the product package by the probe.

61. The method for delivering a product according to claim 48, further comprising:
dividing the sanitizing pouch with a wall to separate a first chemical from a second chemical;
puncturing the wall with the probe to combine the first chemical with the second chemical; and
producing a chemical heat that sanitizes the probe prior to the puncturing of the product package by the probe.

62. The method for delivering a product according to claim 48, further comprising:
sterilizing the probe and the delivery device; and
sealing the probe and the delivery device in a sterilized bag.

63. The method for delivering a product according to claim 62, further comprising puncturing the bag and the sanitizing pouch with the probe to release the sanitizing agent over the probe and the bag thereby sanitizing the bag and the probe prior to the puncturing of the product package by the probe.

64. The method for delivering a product according to claim 63, further comprising puncturing the product package with the probe.

65. The method for delivering a product according to claim 64, further comprising removing the delivery device from the bag.

66. The method for sanitizing a product delivery system according to claim 65, further comprising flowing the sanitizing agent into the delivery device thereby sanitizing the delivery device.

67. The method for delivering a product according to claim 66, further comprising drawing product from the product package utilizing the delivery device.

68. The method for delivering a product according to claim 66, further comprising displacing product from the product package to the delivery device.

69. The method for sanitizing a product delivery system according to claim 66, further comprising flowing the sanitizing agent into a one-way valve disposed in an outlet of the delivery device thereby sanitizing the one-way valve.

70. The method for delivering a product according to claim 69, further comprising delivering the product from the delivery device via the one-way valve disposed in the outlet of the delivery device.

71. The method for sanitizing a product delivery system according to claim 65, further comprising flowing the sanitizing agent into a one-way valve disposed in an inlet of the delivery device thereby sanitizing the one-way valve.

72. The method for sanitizing a product delivery system according to claim 71, further comprising flowing the sanitizing agent into the delivery device thereby sanitizing the delivery device.

73. The method for delivering a product according to claim 72, further comprising drawing product from the product package utilizing the delivery device.

74. The method for delivering a product according to claim 72, further comprising displacing product from the product package to the delivery device.

75. An apparatus for delivering a product, comprising:
a product package;
a delivery device including an inlet thereto and an outlet therefrom;
a probe coupled with the inlet of the delivery device;
a bag, wherein the probe and the delivery device are sterilized and sealed in the bag; and
a patch securable to the bag, the patch impregnated with a sanitizing agent, whereby the puncturing of the bag and the patch by the probe releases the sanitizing agent over the probe thereby sanitizing the probe prior to the puncturing of the product package by the probe.

76. The apparatus for delivering a product according to claim 75, wherein the patch is securable to the bag by an adhesive including a sanitizing agent.

77. The apparatus for delivering a product according to claim 75, wherein the patch is securable to the product package by an adhesive including a sanitizing agent.

78. The apparatus for delivering a product according to claim 75, wherein a cover mounts over the patch to protect the patch from contamination.
